(19) United States
(12) Patent Application Publication
(10) Pub. No.: US 2024/0002804 A1
(43) Pub. Date: Jan. 4, 2024

(54) CULTURED ADIPOSE TISSUE

(71) Applicant: Trustees of Tufts College, Medford, MA (US)

(72) Inventors: David L. Kaplan, Concord, MA (US); Andrew Stout, Cambridge, MA (US); John Yuen, JR., Somerville, MA (US); Natalie Rubio, Medford, MA (US)

(21) Appl. No.: 18/252,627

(22) PCT Filed: Nov. 12, 2021
(86) PCT No.: PCT/US2021/072389

§ 371 Date: May 11, 2023

(51) Int. Cl. C12N 5/077; C12M 1/12; C12N 9/10

(52) U.S. Cl. CPC C12N 5/0653; C12M 25/10; C12N 9/1044; C12N 2506/1346; C12N 2506/45; C12N 2513/00; C12N 2533/56; C12N 2533/74; C12N 2533/78; C12N 2533/54

(57) ABSTRACT

The present disclosure relates to cultured adipose tissue. In one embodiment, the cultured adipose tissue is produced by culturing adipose cells in a culture media in vitro, harvesting the adipose cells after a desired amount of adipose cells are produced, and aggregating the harvested adipose cells to provide the cultured adipose tissue. In some embodiments, aggregating the harvested adipose cells comprises mixing the harvested adipose cells with a hydrogel or binder in a three-dimensional (3D) mold. In other embodiments, aggregating the harvested adipose cells comprises cross-linking the harvested adipose cells in a 3D mold. The cultured adipose tissue have a defined 3D shape and a size on the macroscale. In some embodiments, the cultured adipose tissue may be a food product.

### CROSS-REFERENCE TO RELATED APPLICATIONS

This application is related to, claims priority to, and incorporated by reference herein for all purposes U.S. Provisional Patent Application No. 63/112,738, filed Nov. 12, 2020.

### FIELD OF INVENTION

The present disclosure generally relates to cultured adipose tissue produced on a macroscale level. The present disclosure further relates to methods for producing cultured adipose tissue on a macroscale level without reliance on vascularization or perfusion to maintain cell viability.

### BACKGROUND

Conventional animal agriculture for the production of meat (muscle and fat tissue) is linked to numerous drawbacks such as environmental degradation, zoonic disease emergence, antimicrobial resistance, and animal welfare concerns. To provide the world with alternatives to animal products having reduced negative impacts on animals and the environment, there is increasing interest in producing cultured (in vitro) fat tissue.

In the field of alternative proteins, existing solutions to recapitulate the fat content of meat largely revolve around the direct addition or utilization of plant fats and oils (e.g., coconut oil). Recent developments in this realm include the use of oleogels, where nanoscale globules of vegetable oils are generated to better create the texture of solid fats (i.e., animal fats). However, plant-based fats do not incorporate the often complex aroma and flavor of meat, as well as species-specific flavors that distinguish meat from cows, pigs, chicken, fish, and so on.

Native (in vivo) adipose tissue is largely a dense packing (aggregation) of lipid-filled adipocytes held together by a sparse extracellular matrix (ECM). This is opposed to muscle tissue which is comprised of aligned fibers in a multi-hierarchical structure. To date, three-dimensional (3D) culture has been the main approach for generating bulk/macroscale tissues. These tissue engineering strategies involve the in vitro growth of cells over 3D scaffolds. However, it is challenging to scale up 3D culture due to mass transport limitations with regard to oxygen, nutrients, and waste. It is often quoted in the field that cells cannot remain viable in 3D tissues unless they are within about 200 micrometers of a source of blood or culture media. Overcoming these challenges to maintain cell viability in 3D tissues may require vascularization or the incorporation of an elaborate tissue perfusion system to distribute nutrients to the cells. It is currently infeasible to directly grow large tissues on the macroscale (millimeter scale and up) using contemporary tissue engineering techniques without the use of perfusion or related methods or with the structural features of fat as found in vivo. Likely due to these challenges, large-scale production of adipose tissue that mimics those found in vivo does not appear to have been implemented to date.

Thus, there remains a need for strategies for the large-scale production of cultured adipose tissue. The present disclosure provides a technical solution to this need.

### SUMMARY

Disclosed herein is a method for producing cultured adipose tissue. The method may include growing adipogenic precursor cells in a first culture media, differentiating the adipogenic precursor cells to adipose cells in a second culture media, and harvesting the adipose cells. The method may further include aggregating the harvested adipose cells to provide the cultured adipose tissue. In some embodiments, growing the adipogenic precursor cells and differentiating the adipogenic precursor cells to adipose cells is carried out in a bioreactor.

Further disclosed herein is a method for producing cultured adipose tissue. The method may include growing adipogenic precursor cells in a culture media, and differentiating the adipogenic precursor cells to adipose cells in the culture media. The method may further include harvesting the adipose cells, and aggregating the harvested adipose cells to provide the cultured adipose tissue.

Also disclosed herein is a method for producing cultured adipose tissue. The method may include culturing adipose cells from adipogenic precursor cells in culture media, harvesting the adipose cells after a desired amount of adipose cells are produced, and aggregating the harvested adipose cells to provide the cultured adipose tissue.

Further disclosed herein is cultured adipose tissue. The cultured adipose tissue may include adipose cells embedded in a hydrogel or binder. The cultured adipose tissue may have a 3D shape and a size on the macroscale. In some embodiments, the cultured adipose tissue is a food product.

Further disclosed herein is cultured adipose tissue. The cultured adipose tissue may include adipose cells cross-linked together. The cultured adipose tissue may have a 3D shape and a size on the macroscale. In some embodiments, the cultured adipose tissue is a food product.

### BRIEF DESCRIPTION OF THE DRAWINGS

FIG. 1 is a schematic representation of cultured adipose tissue, in accordance with the present disclosure.

FIG. 2 is a flow chart of steps that may be involved in producing the cultured adipose tissue, in accordance with the present disclosure.

FIG. 3 is a schematic representation of methods of producing the cultured adipose tissue using bioreactors, in accordance with the present disclosure.

FIG. 4 is an image of adipose tissue produced in Example 1 (right) compared to in vivo adipose tissue (left).

### DETAILED DESCRIPTION

Before the present invention is described in further detail, it is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, “adipogenic precursor cells” or “pre-adipocytes” refer to precursor cells capable of differentiating into mature adipose cells. “Adipogenic precursor cells” or “pre-adipocytes” may be used interchangeably throughout the present disclosure. Non-limiting examples of adipogenic precursor cells include stem cells such as pluripotent stem cells (PSCs), mesenchymal stem cells (MSCs), muscle-derived stem cells (MDSCs), and adipose-derived stem cells (ADSCs) (e.g., porcine, bovine, human, avian (chicken), etc.). In addition, transdifferentiated cells can also be utilized. Other adipogenic precursor cells may include, but are not limited to, dedifferentiated fat (DFAT) cells (e.g., porcine, bovine, etc.), preadipocytes (e.g., human, bovine, avian (chicken), murine, etc.), and fibroblasts (e.g., avian (chicken), bovine, porcine, murine, etc.).

As used herein, “adipose cells” are fat cells or adipocytes. “Adipose cells”, “fat cells”, and “adipocytes” may be used interchangeably throughout the present disclosure.

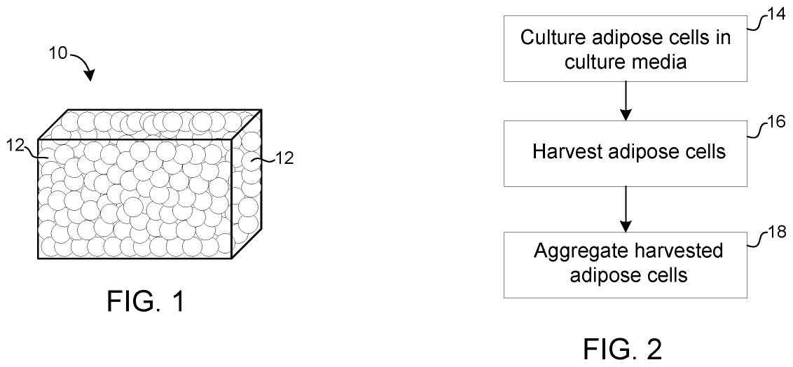

Referring now to the drawings, and with specific reference to FIG. 1, a schematic representation of cultured adipose tissue 10 is shown. The cultured adipose tissue 10 may include adipose cells 12 (or adipocytes 12) in an extracellular matrix. The cultured adipose tissue 10 may be arranged in a defined three-dimensional (3D) shape and may have a size on the macroscale (i.e., millimeter scale and greater). Although a cube-like structure is shown in FIG. 1 for simplicity, it will be understood that the cultured adipose tissue 10 may have any suitable 3D shape in practice. In some embodiments, the cultured adipose tissue 10 may be a food product suitable for consumption. In other embodiments, the cultured adipose tissue 10 may be incorporated as an ingredient in a food product suitable for consumption. As explained further below, the cultured adipose tissue 10 is produced using a method that circumvents the mass transport limitations associated with directly culturing bulk or large scale 3D tissues.

Turning to FIG. 2, a general exemplary method for producing the cultured adipose tissue is shown. At a first block 14, a mass of adipose cells 12 (individual adipose cells 12, or small clusters of adipose cells 12) are cultured from adipogenic precursor cells in culture media. For instance, the block 14 may include growing adipogenic precursor cells to confluency (or to a desired coverage/number of cells on a surface or in suspension) in a first culture media, and then differentiating the adipogenic precursor cells into adipose cells 12 in a second culture media. The first culture media may be an adipogenic induction media which supports proliferation of the adipogenic precursor cells, and the second culture media may be a lipid accumulation media to provide large numbers of lipid-filled adipose cells 12. In alternative embodiments, a single culture medium may be used for both proliferation/growth of the adipogenic precursor cells and for differentiation of the adipogenic precursor cells into adipose cells. The culture time may be tuned to control lipid yield and droplet size. For example, Applicant has found that longer culture times (about a month) yield droplets comparable to in vivo fat (e.g., chicken). In some embodiments, the adipose cells 12 may be genetically modified to improve their growth and lipid accumulation for more efficient scale up.

After the adipose cells 12 have accumulated sufficient lipid and a desired amount of adipose cells 12 are generated, the culture is ended, and the lipid-laden adipose cells 12 are harvested according to a block 16. In some embodiments, the block 16 may include detaching the adipose cells 12 from a substrate, and draining the adipose cells of non-cell liquid.

At a next block 18, the harvested adipose cells 12 may be aggregated in a 3D mold (e.g., a 3D printed mold) having a desired 3D shape to generate the 3D adipose tissue 10. In some embodiments, the block 18 may involve embedding the harvested adipose cells 12 in a hydrogel or a binder in a 3D mold. Suitable hydrogels or binders include, but are not limited to, food safe compounds such as alginate, cellulose, gelatin, mycelium, fibrin, and combinations thereof. In some embodiments, the hydrogel or binder is alginate which is a material used as a fat replacer in the food industry. For instance, the block 18 may include mixing the harvested and drained adipose cells 12 with an alginate solution at a specified volumetric ratio in the 3D mold.

In some aspects, the block 18 may involve cross-linking the harvested adipose cells 12 in a 3D mold. In some aspects, cross-linking the harvested adipose cells includes enzymatically cross-linking the harvested adipose cells using transglutaminase.

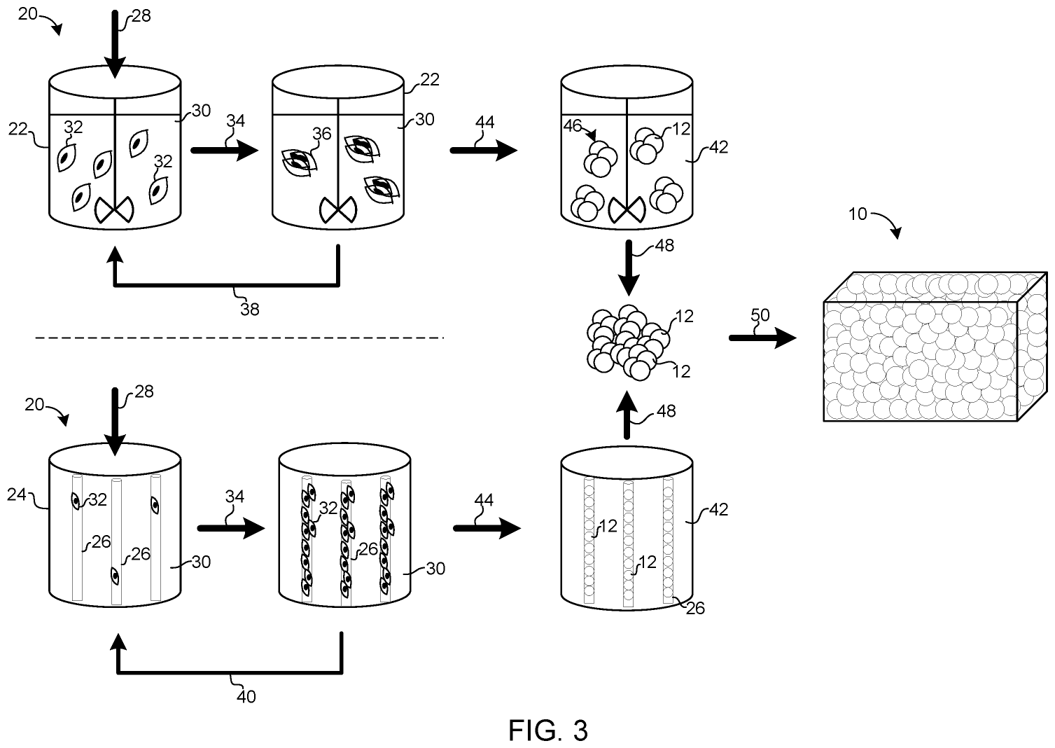

FIG. 3 shows scalable processes for the mass production of the cultured adipose tissue 10. The processes may be carried out in a bioreactor 20, such as a stirred suspension tank bioreactor 22 (top) or a hollow fiber bioreactor 24 having hollow fiber membranes 26 (bottom). Other types of bioreactors apparent to those skilled in the art may also be used and are within the scope of the present disclosure such as, but not limited to, stirred tank bioreactors, vertical wheel bioreactors, airlift bioreactors, and packed bed bioreactors. Production of the adipose tissue 10 in the bioreactor 20 may involve seeding 28 a first culture media 30 (adipogenic induction media) in the bioreactor 20 with adipogenic precursor cells 32. The adipogenic precursor cells 32 may then proliferate 34 to confluency (or to a desired coverage/number of cells on a surface or in suspension) in the bioreactor 20. In some embodiments, the adipogenic precursor cells 32 may form small aggregates or spheroids 36 as they proliferate (see FIG. 3, top). The spheroids 36 may be dissociated 38 into single adipogenic precursor cells 32 and allowed to proliferate 34 further (see FIG. 3, top). In the hollow fiber reactor 24, the adipogenic precursor cells 32 may proliferate on the surface of the hollow fiber membranes 26 (see FIG. 3, bottom). In this case, the adipogenic precursor cells 32 may be detached 40 from the hollow fiber membranes 26, and the detached adipogenic precursor cells 32 may be used to re-seed the media 30 for further proliferation 34.

As the first culture media 30 is changed to a second culture media 42 (the lipid accumulation media), the cells may accumulate lipids and differentiate 44 into adipose cells 12. The adipose cells 12 may grow separately or in small clusters 46 (see FIG. 3, top). In the hollow fiber bioreactor 24, the adipose cells 12 may develop on the surface of the hollow fiber membranes 26. In some embodiments, a single culture medium may be used for both proliferation 34 and differentiation 44. After the adipose cells 12 have grown and accumulated sufficient lipid, the adipose cells 12 may be harvested 48. In the hollow fiber bioreactor 24, the harvesting may involve detaching the adipose cells 12 from the hollow fiber membranes 26. The harvested adipose cells 12 may then be aggregated 50 in a 3D mold to provide the cultured adipose tissue 10. As explained above, suitable methods for binding and aggregating 50 the adipose cells 12 include cross-linking (e.g., enzymatic cross-linking with transglutaminase), as well as embedding the adipose cells 12 in hydrogels such as alginate.

The technology disclosed herein provides a novel and scalable approach to cultured fat generation. The present disclosure leverages large-scale cell proliferation and scale up technology to generate a required amount of in vitro adipose cells, after which the cells are aggregated or packed into a solid 3D construct on the macroscale. The adipose cells can be cultured in bioreactors with easy access to the culture media, followed by aggregation into macroscale 3D tissues after sufficient adipocyte maturation. The aggregation of adipocytes or adipocyte clusters recapitulates native fat tissue from a sensory perspective as adipose tissue in vivo is largely a dense aggregation of lipid filled adipocytes with a sparse extracellular matrix.

Additionally, the method of the present disclosure produces bulk cultured adipose tissue in a way that circumvents the mass transport limitations associated with directly culturing or engineering large 3D tissues. Aggregation at the end of cell culture removes the need for nutrient delivery to the adipose cells via vascularization or an elaborate tissue perforation system. This is because, for food applications, the cultured adipose cells do not need to stay alive once formed into the final edible tissue. This is analogous to meat production in conventional animal agriculture where muscle and fat cells gradually cease to be viable after slaughter. In contrast, for medical applications, cells in 3D tissues may be expected to remain viable to be used for implantation into the body or for testing in an in vitro tissue model. Accordingly, the adipose tissue production method of the present disclosure is less costly than other methods that rely on complex perfusion and mixing systems to distribute nutrients during cell growth.

According to the methods of the present disclosure, monocultures of adipocytes and pre-adipocytes may be sufficient for the production of large fat droplets without the need for supporting cell types. Standard cell culture conditions are sufficient for the type of adipocyte culture outlined in this disclosure, and no specific coatings on tissue culture plastics were required to achieve desired adipocyte growth and development. Furthermore, the pre-adipocytes and adipocytes of various livestock species may be grown in serum-free culture media according to the present disclosure, thereby eliminating a major obstacle in in vitro fat culture. These advantages further help reduce production costs. Co-cultures can also be considered for enhanced fat outcomes, such as the use of fibroblasts or muscle cells in the cultures, such as to increase the quality of the fat products or to alter the texture and composition.

The cells, tissues, adipogenic precursor cells, adipose tissue, adipose cells, and/or adipogenic tissue disclosed herein can be from an animal source, including, without limitation, from bovine, avian (e.g., chicken, quail), porcine, seafood, or murine sources. The cells, tissues, adipogenic precursor cells, adipose tissue, adipose cells, and/or adipogenic tissue may be derived from seafood such as fish (e.g., salmon, tuna, tilapia, perch, mackerel, cod, sardine, trout, etc.), shellfish (e.g., clams, mussels, and oysters); crustaceans (e.g., lobsters, shrimp, prawns, and crayfish), and echinoderms (e.g., sea urchins and sea cucumbers).

### EXAMPLES

### Example 1

Broadly speaking, the two methods outlined below for generating macroscale, in vitro fat tissue involve proliferating adipocytes and inducing their accumulation of lipids under conditions where the cells are grown separated from each other, or in small clusters. This addresses mass transport limitations that would be present if one were to simply culture a dense, macroscale aggregation of adipocytes as-is. Here, once a large population of individual lipid laden adipocytes (or small clusters of lipid laden adipocytes) are grown, the cells are densely aggregated to recapitulate in vivo fat, culminating in the final macroscale adipose tissue construct.

In the first approach, hollow fiber bioreactors were used to proliferate preadipocytes or other proliferative stem cell precursors. The fibers were seeded at low density and allowed to multiply until they reach a higher density that still maintains the cells in a proliferative state. Cells were then detached and reseeded at low density into more bioreactor modules. Fibers can be seeded on the inside, outside, or on both surfaces to maximize cell density per bioreactor volume. Once appropriate amounts of biomass were produced, cells could be grown in the same hollow fiber bioreactor configuration with adipogenic differentiation culture media driving fat accumulation (e.g fatty acid or thiazolidinedione-based differentiation). After lipid accumulation, cells were detached from the fibers and aggregated to form cultured fat tissue.

The second approach involved using suspension culture bioreactors, with cells added as a single suspension and proliferated to form clusters of suspended adipocyte precursor cells. Suspension bioreactors include (but are not limited to) stirred tank, vertical wheel, airlift, packed bed bioreactors. To continue proliferation, clusters were collected, dissociated, and reseeded as a single cell suspension. For adipogenesis, cells were grown into clusters and induced to halt proliferation and begin lipid accumulation via a culture media change (e.g fatty acid or thiazolidinedione-based differentiation). Lipid-laden adipocyte clusters were then aggregated to form cultured fat.

Methods for the final aggregation of lipid laden adipocytes include, but are not limited to, centrifugation, centrifugation followed by transglutaminase cross-linking, and cell seeding into a hydrogel or other scaffold that closely matches the mechanics of the extracellular matrix typically present in adipose tissue. Examples of scaffold materials include, but are not limited to: cellulose, alginate, mycelium, and gelatin of microbial origin or upcycled gelatin waste/by-products.

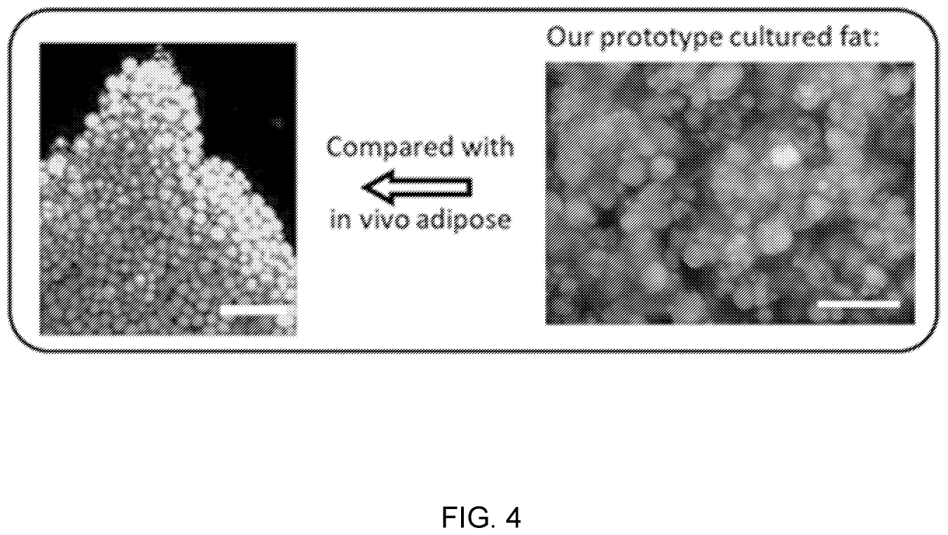

FIG. 3 (bottom) outlines the invention with stem cells proliferated and differentiated into adipocytes via a hollow fiber bioreactor-based approach. FIG. 3 (top) outlines the invention using stirred suspension tanks as the bioreactor for proliferating stem cells and differentiating adipocytes as clusters or small spheroids. FIG. 4 shows an example of in vitro cultured adipocytes embedded in a fibrin hydrogel, with a comparison to in vivo fat.

Cultured meat promises a potential alternative to meat produced from animal agriculture that is more environmentally friendly, better for animal welfare, while combating antibiotic resistance and zoonotic disease transmission. Cultured meat mostly consists of muscle and fat tissue. Fat plays a crucial role in the flavor, and texture of meat, but there are currently no methods for the large scale generation of adipose cells and tissue for cultured fat. It is currently infeasible to directly grow large tissue constructs due to limitations on cell survival and growth in the absence of a complex and elaborate network to perfuse nutrition and oxygen. In the body this is addressed by the circulatory system—a hierarchical arrangement of arteries, arterioles, capillaries, venules and veins with most cells lying within 200 μm of a blood vessel.

Here, we present techniques for circumventing the need for a complex system of nutrient perfusion within macroscale tissues by growing mature adipocytes individually or as small clusters, ultimately aggregating them into a macroscale tissue construct/cultured fat. Vasculature is unnecessary in the final tissue as the cells within the cultured fat construct do not need to be kept alive. Using existing bioreactor and biomanufacturing techniques in a novel manner, this invention enables the realization of the various benefits producing cultured fat might have over conventional methods based on animal agriculture.

What is claimed:

1. A method for producing cultured adipose tissue, comprising:
growing adipogenic precursor cells in a first culture media;
differentiating the adipogenic precursor cells to adipose cells in a second culture media;
harvesting the adipose cells; and
aggregating the harvested adipose cells to provide the cultured adipose tissue.

2. The method of claim 1, wherein growing the adipogenic precursor cells in the first culture media comprises seeding the adipogenic precursor cells into a bioreactor containing the first culture media, and allowing the adipogenic precursor cells to proliferate in the bioreactor.

3. The method of claim 2, wherein differentiating the adipogenic precursor cells to adipose cells comprises changing the first culture media to the second culture media in the bioreactor.

4. A method for producing cultured adipose tissue, comprising:
growing adipogenic precursor cells in a culture media;
differentiating the adipogenic precursor cells to adipose cells in the culture media;
harvesting the adipose cells; and
aggregating the harvested adipose cells to provide the cultured adipose tissue.

5. The method of claim 1 or 4, wherein the method is carried out in a bioreactor.

6. The method of claim 2, 3 or 5, wherein the bioreactor is a stirred suspension tank bioreactor.

7. The method of claim 2, 3 or 5, wherein the bioreactor is a hollow fiber bioreactor.

8. A method for producing cultured adipose tissue, comprising:
culturing adipose cells from adipogenic precursor cells in culture media;
harvesting the adipose cells after a desired amount of adipose cells are produced; and
aggregating the harvested adipose cells to provide the cultured adipose tissue.

9. The method of any one of the preceding claims, wherein the adipogenic precursor cells are pluripotent stem cells.

10. The methods of any of claims 1 to 8, wherein the adipogenic precursor cells are mesenchymal stem cells.

11. The method of any one of the preceding claims, wherein aggregating the harvested adipose cells comprises mixing the harvested adipose cells with a hydrogel or binder in a 3D mold.

12. The method of claim 11, wherein the hydrogel or binder is selected from the group consisting of alginate, cellulose, gelatin, mycelium, fibrin, and combinations thereof.

13. The method of any one of the preceding claims, wherein aggregating the harvested adipose cells comprises mixing the harvested adipose cells with alginate.

14. The method of any of claims 1 to 10, wherein aggregating the harvested adipose cells comprises cross-linking the harvested adipose cells in a 3D mold.

15. The method of claim 14, wherein cross-linking the harvested adipose cells in the 3D mold comprises enzymatically cross-linking the harvested adipose cells using transglutaminase.

16. The method of any one of the preceding claims, wherein the cultured adipose tissue has a size on the macroscale.

17. The method of any one of the preceding claims, wherein the cultured adipose tissue has a defined 3D shape.

18. Cultured adipose tissue comprising adipose cells embedded in a hydrogel or binder, wherein the cultured adipose tissue has a three-dimensional (3D) shape and a size on the macroscale.

19. The cultured adipose tissue of claim 18, wherein the hydrogel or binder is selected from the group consisting of alginate, cellulose, gelatin, mycelium, fibrin, and combinations thereof.

20. Cultured adipose tissue comprising adipose cells cross-linked together, wherein the cultured adipose tissue has a three-dimensional (3D) shape and a size on the macroscale.

21. The cultured adipose tissue of claim 20, wherein the adipose cells are cross-linked with transglutaminase.

22. The method or cultured adipose tissue of any one of the preceding claims, wherein the cultured adipose tissue is a food product.

23. The method or cultured adipose tissue of any one of the preceding claims, wherein the cultured adipose tissue is an ingredient of a food product.

24. The method or cultured adipose tissue of any one of the preceding claims, wherein one or more components of the adipose tissue are ingredients in a food product.

25. The method or cultured adipose tissue of any one of the preceding claims, wherein the cultured adipose tissue is produced without perfusion.
